(19) United States
(12) Patent Application Publication
(10) Pub. No.: US 2011/0189732 A1
(43) Pub. Date: Aug. 4, 2011

(54) Process for the Fermentative Production of Erythropoietin

(75) Inventors: Wolfgang Wienand, Bergisch-Gladbach (DE); Franz-Rudolf Kunz, Gelnhausen (DE); Dietmar Reichert, Eschau (DE); Wilfried Eul, Alzenau (DE); Rudolf Hanko, Düsseldorf (DE); Christian Birr, Neckargemund (DE); Monika Singhofer-Wowra, Ladenburg (DE); Dagmar Schopohl-König, Dresden (DE); Lars Faber, Ober-Ramstadt (DE)

(73) Assignee: EVONIK DEGUSSA GMBH, Essen (DE)

(21) Appl. No.: 12/996,070

(22) PCT Filed: Jun. 3, 2009
(86) PCT No.: PCT/EP2009/056820

§ 371 Date: Feb. 5, 2011

(30) Foreign Application Priority Data
Jun. 4, 2008 (DE) 10 2008 002 210.1

(51) Int. Cl. C12P 21/00

(52) U.S. Cl. 435/69.4

(57) ABSTRACT

The present invention relates to a process for the fermentative continuous production of erythropoietin, where eukaryotic erythropoietin-producing cells are cultured in a perfusion reactor while retaining the cells, the glucose concentration in the culture supernatant being adjusted via the perfusion rate and the cell number via the cell retention rate and/or the outward transferal of defined amounts of cell-containing culture medium from the bioreactor within preset zones.

The present invention relates to a process for continuous fermentative production of erythropoietin (EPO). The process is characterized in that it is carried out in a perfusion reactor with cell retention, with the fermentation process being controlled only by a few selected measurement and control parameters so as to influence both the productivity of the chosen host organism in respect of EPO and EPO product quality in an advantageous manner.

Erythropoietin, EPO for short, is a glycoprotein which stimulates the formation of erythrocytes in the bone marrow. EPO is mainly produced in the kidneys and reaches its target site from there via the circulation. In kidney failure, the damaged kidneys produce too little EPO or no EPO at all, resulting in too few erythrocytes being produced from the stem cells of the bone marrow. This “renal anemia” can be treated by administering physiological amounts of EPO that stimulate the formation of erythrocytes in the bone marrow. The therapeutic action and use of EPO is described in detail, for example, in Eckardt K. U., Macdougall I. C., Lancet 2006, 368, 947-953, Jelkmann W.; Physiol. Rev. 1992, 72, 449-489, Eschbach J. W. et al., N. Engl. J. Med., 1987, 316, 73-78, EP-B 0 148 605, EP-B 0 209 539, EP-B 0 205 564, Huang S. L., PNAS 1984, 2708-2712, Lai, P. H., et al., J. Biol. Chem. 1986, 261, 3116-3121, and in Dietzfelbinger H. et al., Manual Supportive Maβnahmen and symptomorientierte Therapie, Tumorzentrum Munich, Germany, 2001, 70-77.

The EPO used for administration can either be obtained from human urine or be prepared by genetic engineering methods. Since the human body contains only very small amounts of EPO, isolating EPO from the natural source for therapeutic uses is virtually impossible. Consequently, genetic engineering methods offer the only economically feasible way of producing this substance in relatively large quantities.

Erythropoietin can be produced recombinantly since the human erythropoietin gene was identified in 1984. Since the beginning of the 1990s, various medicaments have been developed which contain human erythropoietin produced biotechnologically in eukaryotic cells modified by genetic recombination. EP-A-0 148 605 and EP-A-205 564, inter alia, describe the production of recombinant human erythropoietin.

The “degree of sialylation”, i.e. the content of sialylic acids terminally linked to the protein via sugar chains, is of crucial importance for the effectiveness of some proteins such as erythropoietin or interferon, for example. Proteins with a higher degree of sialylation usually have a higher specific activity. According to the prior art, those proteins such as, for example, EPO, t-PA (tissue plasminogen activator) or blood clotting factor VIII, whose activity depends inter alia on their degree of sialylation, are produced in cultures of mammalian cells which are capable of such as necessary, post translational glycosylation or sialylation of the protein. Usually, EPO is recombinantly produced in Chinese hamster ovary (CHO) host cells. While the latter have previously been cultured in culture media supplemented with fetal calf serum and sometimes also bovine insulin, they are nowadays cultured regularly in serum- and protein-free medium. This eliminates the risk of contaminations with bovine proteins, bovine viruses, bovine DNA or other undesired substances. The skilled worker is familiar with the ingredients of such serum- and protein-free culture media. They consist of a mixture of amino acids, fatty acids, vitamins, inorganic salts and hormones in different concentrations, as specified, for example, in EP-B1 481 791 and WO88/00967 A1. The culture medium here has considerable influence on the growth rate, cell density, translation and transcription of the host cells and therefore, inter alia, also on the glycosylation and sialylation pattern of the recombinantly produced protein. Thus, serum-free media are normally used that are supplied by various manufacturers, for example the MAM-PF2 medium (sold inter alia by Bioconcept, Allschwil, Switzerland) or the DMEM and DMENU12 media (supplied, for example, by Invitrogen/Gibco, Eggenstein, Germany).

In principle, three different procedures for culturing host cells for producing recombinant proteins such as, for example, EPO can be distinguished:

In a batch process, medium and cells are introduced into the bioreactor at the start of cultivation. Until the end of the cultivation, there are neither nutrients added nor cells removed from the fermenter, and only oxygen is fed in. Once one or more substrates are exhausted, the process is stopped and the products are harvested from the fermentation supernatant. A variation of this batch process is the “repeated batch process” which involves leaving part of the culture volume for inoculation in the bioreactor at the end of the fermentation, filling the reactor with new medium and restarting the fermentation process.

An advantage of the batch process is its simple technical implementation. Disadvantageously, however, the capacity of the cells for producing the recombinant proteins is not fully utilized in general due to selective depletion of nutrients in the culture medium and to accumulation of metabolic products which are toxic for the cells, such as ammonium and lactate, for example. Another disadvantage is the fact that the product accumulating in the batch fermenter is constantly exposed to the metabolic enzymes which likewise accumulate, and this may have an adverse influence on product quality and/or product yield. As described in Gramer M. J. et al., Biotechnology 1995, 13, 692-698, this applies specifically to the “sialidases” which are capable of removing the terminal sialylic acids from already formed glycoproteins and, as a result, lower the yield of desired highly sialylated glycoprotein. Another disadvantage of the batch process is the unfavorable ratio between production time which is limited due to the limited supply of nutrients for the cell culture (typically around 5 to 10 days) and the total time of the cycle, which additionally includes the time for setting up, cleaning and sterilizing the bioreactor (typically around up to 4 days).

The second known cultivation process is the continuous process in which fresh medium is continuously fed in and fermenter contents are removed to the same extent. This results in a continuous supply of nutrients, and at the same time undesired metabolic products such as the growth-inhibiting substances ammonium and lactate are removed or diluted. Consequently, higher cell densities can be obtained and maintained over a comparatively long period of time by this process. A special case of the continuous process type comprises “dialysis reactors”, with which high molecular weight substances such as proteins are retained in the fermenter, while low molecular weight substances such as substrates can be added or the major waste products ammonium and lactate can be removed from the system. Apart from these advantages of the continuous process, there are disadvantages, in particular in respect of the comparatively increased risk of contamination by contaminations of the cell retention systems (typically membrane filters that are difficult to clean) and the deposition of cell material on the filter surface during the process which can reduce the flow up to a complete blockage of the membrane. An alternative is offered by ultrasound-supported retention systems in which the cells are prevented from escaping the fermentation reactor by an ultrasonic standing wave and the use of membrane filters is no longer needed.

The use of perfusion reactors in microbial production of chemical compounds and proteins with various cell retention systems is well known and has been described for EPO too (BioProcess International 2004, 46; Gorenflo et al., Biotech. Bioeng. 2002, 80, 438; www.sonosep.com/biosep.htm; WO 95/01214).

In this context, for example, von Wang M. D. et al., Biotechnology and Bioengineering 2002, 77 (2), 194-203 discuss various systems in which the cells are bound to macroporous beads and, due to the pores, can colonize large surfaces, resulting in high cell densities. Nutrients are supplied by continuously feeding fresh medium, with the cell-covered beads being washed to the top at the same time. It is also possible to pack a stirred tank with small polyester disks (approx. 1 cm in diameter) rather than with beads and to continuously pass fresh medium through them (Jixian D. et al., Chinese Journal of Biotechnology 1998, 13 (4), 247-252). This, however, requires the cells to grow in an adherent manner. Colonizing supports, regardless of their type and chemical composition, has the disadvantage that the cells can grow therein so densely that the inner layers can no longer be supplied properly and the cells colonized there stop production and/or also release undesired metabolites to such an extent that they are not removed adequately and therefore can adversely influence product quality.

The third possible process, finally, is fed batch fermentation which comprises starting the cultivation in a fermenter which has been filled only partly with culture medium and, after a short growing phase, little by little adding fresh medium. This makes higher cell densities and longer process times than in the batch process possible. Another advantage of this process is the fact that the metabolism of the cells can be influenced via the extent of feeding, which may result in a lower production of waste substances. Compared with the continuous process, the product of the cells here is accumulated in the fermenter over a longer period of time, thereby achieving higher product concentrations, and this facilitates subsequent work-up. This requires, however, the product of the cells to be stable enough for it not to be degraded enzymatically or to be decomposed in another way when it is in the fermenter for several days. Another disadvantage of this process is the fact that the physiological conditions in the bioreactor can change disadvantageously due to concentrated substrate solutions being fed in.

A major problem of the culturing of mammalian cells is that of supplying the cells with sufficient nutrients, without the degradation products of said nutrients accumulating beyond a limit critical for cellular physiology. The main energy sources used by animal cells are glucose and glutamine, whose major degradation products, lactate and ammonium, respectively, at relatively high concentrations, inhibit growth and metabolism of the cells and result in cell death (Hassell et al., Applied Biochemistry and Biotechnology 1991, 30, 29-41). When culturing animal cells, it is therefore advantageous to reduce accumulation of lactate and ammonium while supplying sufficient amounts of nutrients, in order to thus achieve higher cell densities and a higher product yield.

One possible way of reducing the production of waste products is that of adding substrates in a controlled way, which is also referred to as “catabolic control”. This utilizes the dependence of the cellular metabolism on the concentrations of nutrients provided in the fermentation medium. A limiting feed of glutamine and/or glucose was shown to result in a markedly reduced production of ammonium and lactate in hybridoma cells (Ljunggren & Häggström, Biotechnology and Bioengineering 1994, 44, 808-818).

In fed batch cultures in which the glucose concentration had been adjusted by adding concentrated medium in a controlled way, an alteration of the cellular metabolism was observed after some time, which is also referred to as metabolic shift. This metabolic shift is induced by limiting the glucose and glutamine concentrations in the medium over several days and results in fewer nutrients being taken up and metabolized by the cells. Subsequently, the glucose and glutamine contents in the culture medium increase, whereas production of the waste substances lactate and ammonium falls markedly due to the reduced consumption (Zhou et al., Biotechnology and Bioengineering 1995, 46, 579-587). Said metabolic shift was observed not only in hybridoma cells but also in cell lines such as SPO, HEK-293, BHK and CHO.

The metabolic shift can achieve high cell densities of more than 10⁷ cells per milliliter accompanied by comparatively long process times, since the waste products lactate and ammonium do not accumulate at concentrations that adversely regulate cell growth. It is important here that the feeding solution is adapted to the requirements of the cells in order to ensure achieving the metabolic shift and to prevent both exhaustion and excessive accumulation of particular nutrients and consequently a strong increase in osmolality (Xie and Wang, Biotechnology and Bioengineering 1994, 43, 1175-1189 and Biotechnology and Bioengineering 1996, 51, 725-729). It is furthermore important to provide the cells nevertheless with enough glucose as substrate for glycosylation of the recombinant proteins.

To enable the concentrations of the supplied nutrients to be adjusted to the consumption by the cells, normally complicated processes are employed in order to determine the current consumption by the cells and subsequently to adjust the rate of feeding as required. This includes inter alia measuring the glucose concentration, for example by flow injection analysis, or measuring the consumption of oxygen by the cells.

Thus, for example, U.S. Pat. No. 6,180,401 discloses a fed batch cell culture process in which the glucose concentration is measured continuously and is kept within a certain range in the culture medium by adjusting the feeding as a function of the measured data. According to the teaching of US 2002/0099183 too, the rate of feeding in glucose is determined via the glucose concentration, thereby keeping said glucose concentration in the culture medium within a particular range.

EP-A-1 036 179 describes, on the basis of a fed batch process, an addition of nutrients as needed as a function of the glucose concentration in the culture medium.

WO 97/33973 discloses a culturing process which involves measuring production of an electrically charged metabolic product on the basis of the conductivity of the medium and adapting the feeding rate accordingly.

U.S. Pat. No. 5,912,113 describes a fermentation process for microorganisms which involves feeding every time that the carbon source in the medium is exhausted and, as a result, an increased pH or an increased concentration of dissolved oxygen is measured in the medium.

Aside from technical process parameters such as the nutrient supply by way of the media composition, as discussed above, cell line-specific properties which may be expressed as different growth rates, production kinetics, cell vitality, posttranslational processing for glycosylation and sialylation, also play a central part in terms of product quality of the EPO obtained and the overall productivity of the fermentation process. For example, it is known, as described in Lloyd D. R. et al., Cytotechnology 1999, 30, 49-57, that, depending on the life cycle phase the producing host cell is in, the cellular metabolism may be substantially different and, as a result in the present case, EPO may be produced in different amounts and quality, in particular with regard to the degree of glycosylation and sialylation.

Since fermentative production of EPO in eukaryotic cells is very expensive due to the complicated processes illustrated, the typically comparatively low product concentrations in the fermentation supernatant, and the use of protein- and serum-free culture media with high price components, the development of more efficient production processes is of considerable importance.

The technical problem addressed by the present invention was therefore that of developing a process for fermentative production of erythropoietin, which has advantages over the processes of the prior art both with regard to the simplicity of process management and with regard to the yield of high quality erythropoietin. The EPO obtained should meet all requirements of the official standard and in particular all requirements with regard to isoform composition and glycosylation and sialylation patterns (Ph. Eur. 04/2002:1316).

The technical problem is solved by a process for continuous fermentative production of erythropoietin, which process comprises culturing eukaryotic EPO-producing cells in a perfusion reactor with retention of the cells, wherein the glucose concentration in the reactor is adjusted via the rate of perfusion of the culture medium and the number of cells in the reactor is adjusted via the rate of cell retention, in each case within a predetermined range.

Advantageously, the inventive continuous process for fermentative production of erythropoietin comprises adjusting

a) the rate of perfusion of the culture medium (as control parameter) as a function of the glucose concentration in the fermentation reactor (as measurement parameter), and
b) the cell retention rate of the cell retention device (as control parameter) as a function of the cell density in the fermentation reactor (as measurement parameter)
in a suitable manner within predetermined ranges.

As an alternative to or in combination with b), it is also possible to export, as required, defined amounts of cell-containing culture medium out of the bioreactor at intervals and to achieve in this way a particular cell density in the reactor.

The other relevant process parameters such as, for example, pH, temperature, oxygen partial pressure, stirring speed and composition of the supplied medium, are preferably kept constant over the entire fermentation period.

The process illustrated combines in a novel way various measures of increasing both the product yield and the product quality of erythropoietin:

(1) Perfusion ensures that both cytotoxic metabolic products are continuously exported and fresh nutrients are continuously supplied so as to achieve very high cell densities in the bioreactor and for the cells to be productive over a very long period of time.
(2) To reduce the consumption of expensive culture medium, which is typically very high in perfusion processes, and to make possible an economically improved production process, the rate of perfusion is furthermore chosen such that the glucose content in the culture supernatant firstly does not fall below a lower limit required for efficient cell growth but secondly is limited in such a way that the metabolic shift happens in the cellular metabolism, and the toxic metabolites lactate and ammonium are produced only in reduced amounts and thus need to be exported with only small amounts of fresh medium during perfusion.
(3) By suitably adjusting the cell retention rate of the controllable cell retention system and/or by repeatedly exporting defined amounts of cell-containing medium it is moreover possible to generate in the fermentation solution a group of cells with an increased relative proportion of those cells whose growth has not yet leveled off but which are still in the exponential growth phase and are capable in a specific way of producing a high quality EPO meeting the official standard.

The measures mentioned, setting a suitable cell retention rate and regularly exporting defined amounts of cell-containing culture medium and simultaneously setting a suitable rate of perfusion, act synergistically, and therefore surprisingly an erythropoietin can be obtained in very high yields over an extremely long period of time by a process which, for a continuous process, is very simple and can readily be carried out technically, which erythropoietin has an extremely high proportion of that EPO which meets the legal requirements for medicaments, in particular with regard to its degree of glycosylation and sialylation and distribution of the isoforms.

According to the invention, the glucose concentration and the number of cells can be measured and/or monitored continuously or at particular time points. Preference is given to adjusting the glucose concentration and the cell number continuously. The glucose concentration is adjusted via the rate of perfusion, i.e. by adding fresh culture medium containing glucose as a function of the glucose concentration in the fermentation reactor.

In a preferred process as claimed in claim 1, the glucose concentration in the culture supernatant is adjusted within a range from 0.05 to 1.5 g/l, and the number of cells is adjusted within a range from 0.5×10⁷ to 5.0×10⁷ cells/ml.

Preference is furthermore given to the eukaryotic erythropoietin-producing cells being mammalian cells, preferably human cells and particularly preferably Chinese hamster ovary (CHO) cells.

In a further preferred process, the cells are retained using an ultrasound cell retention system which can preferably be controlled with continuous adjustment.

Preference is furthermore given to the process parameters pH, temperature, oxygen partial pressure, stirring speed and media composition being kept constant within the range of technical deviations over the entire fermentation period.

In a particularly preferred embodiment of the process of the invention, productivity is at least 10, preferably at least 20, more preferably at least 25, and very particularly preferably at least 30, mg of erythropoietin/l of fermentation supernatant. The average productivity is preferably at least 10, preferably at least 15, mg of erythropoietin/1 of fermentation supernatant.

Preference is furthermore given to the average specific productivity per cell and day being at least 0.5 pg, more preferably at least 1.0 pg, and particularly preferably at least 1.2 pg, and very particularly preferably at least 1.4 pg, of erythropoietin.

The average vitality of the cells in the use of the process of the invention is at least 70%, preferably at least 75%, particularly preferably at least 80%, further preferably at least 90%, and very particularly preferably at least 95%.

The process of the invention is carried out preferably with a rate of perfusion during fermentation of between 0.5 and 3, preferably between 1 and 2.5 and particularly preferably between 1.5 and 2.0.

The process according to the present invention is advantageously carried out over a period of at least 10, preferably of at least 20, particularly preferably of at least 30, days and very particularly preferably of at least 40 days.

In a further embodiment of the process, the glucose concentration in the culture supernatant is preferably adjusted within a range from 0.25 to 1.25 g/l, and particularly preferably from 0.5 to 1.0 g/l.

The number of cells in the bioreactor is preferably adjusted within a range from 1.0×10⁷ to 4.0×10⁷ cells/ml, and particularly preferably in a range from 1.5×10⁷ to 3.0×10⁷ cells/ml of fermentation medium.

### DETAILED DESCRIPTION OF THE INVENTION

### Best Embodiment

The present invention relates to a process for continuous fermentative production of erythropoietin, with eukaryotic EPO-producing cells being cultured in a perfusion reactor with retention of the cells, wherein the glucose concentration in the culture supernatant is adjusted via the rate of perfusion and the number of cells is adjusted via the cell retention rate of a cell retention device and/or regular export of defined amounts of cell-containing culture medium, in each case within a predetermined range.

By adjusting according to the invention, in a continuous process for fermentative production of erythropoietin, preferably by means of CHO cells, in a perfusion reactor with a continuously adjustable ultrasound cell retention system

a) the rate of perfusion of the culture medium (as control parameter) as a function of the glucose concentration in the fermentation reactor (as measurement parameter), and
b) the cell retention rate of the cell retention device (as control parameter) as a function of the cell density in the fermentation reactor (as measurement parameter)
in a suitable manner to one another within predetermined ranges, a group of cells is obtained in the fermentation solution, which has an increased relative proportion of cells whose growth has not yet leveled off but which are still in the exponential growth phase. Such cells are in a specific way capable of producing a high quality EPO meeting the official standard.

Both measures, setting a single cell retention rate with simultaneously setting a suitable rate of perfusion, act synergistically and, as a result, an EPO can be obtained in surprisingly high yields of up to 30 mg/l of fermentation supernatant in a process which is surprisingly simple for a continuous process and can readily be carried out technically, which EPO has an extremely high proportion of that EPO which meets the legal requirements for medicaments, in particular in respect of its degree of glycosylation and sialylation and distribution of isoforms.

By adjusting the rate of perfusion, a glucose content in the culture supernatant of from 0.05 to 1.5 g/l, preferably from 0.25 to 1.25 g/l, and particularly preferably from 0.5 to 1.0 g/l, has proved to be advantageous for the present CHO cell line. The rate of perfusion is controlled according to the glucose content measurements in the reactor. In combination therewith, the cell density in the reactor is kept within a range from 0.5×10⁷ to 5.0×10⁷ cells/ml by appropriately adjusting ultrasonic cell retention and regularly exporting defined amounts of cell-containing culture medium. Further preference is given to a cell density of from 1.0×10⁷ to 4.0×10⁷ cells/ml and very preferably a cell density of from 1.5×10⁷ to 3.0×10⁷ cells/ml. Such parameter adjustment is controlled by means of cell density measurements in the reactor.

According to the invention, all other relevant process parameters such as, for example, pH, temperature, oxygen partial pressure, stirring speed, and media composition, are preferably kept constant over the entire fermentation period.

Cultivation is preferably carried out in a serum- and protein-free medium. The skilled worker is familiar with the ingredients of such serum- and protein-free culture media. They consist of a mixture of amino acids, fatty acids, vitamins, inorganic salts and hormones at different concentrations, as specified, for example, in E P-B1 481 791 and WO88/00967 A1. The culture medium here has considerable influence on the growth rate, cell density, translation and transcription of the host cells and therefore, inter alia, also on the glycosylation and sialylation patterns of the recombinantly produced protein. The present invention makes use of serum-free media as supplied by various manufacturers, for example the MAM-PF2 medium (sold inter alia by Bioconcept, Allschwil, Switzerland), the DMEM and DMENU12 media (supplied, for example, by Invitrogen/Gibco, Eggenstein, Germany) or the HyQPF CHO Liquid Soy medium (supplied inter alia by HyClone/Perbio, Bonn, Germany).

The EPO produced according to the invention is preferably recombinant human erythropoietin, produced in eukaryotic cells. Said recombinant EPO is preferably produced in mammalian cells, particularly preferably in human cells and very particularly preferably in CHO cells, as described generally, for example, in EP-A-0 205 564 and EP-A-0 148 605.

Erythropoietin (EPO) means for the purposes of the present invention any protein which is capable of stimulating the production of erythrocytes in bone marrow and which can unambiguously be identified as erythropoietin by the assay described in the European Pharmacopoeia (Ph. Eur. 04/2002:1316) (determining the activity in polycythemic or normocythemic mice). The EPO may be the human wild-type erythropoietin or a variant thereof having one or more amino acid substitutions, deletions or additions. If it is a variant of EPO, then preference is given to said variant differing from human wild-type erythropoietin, due to amino acid substitutions, deletions or additions, only in 1 to 20, preferably in only 1 to 15, particularly preferably in only 1 to 10, and very particularly preferably in only 1 to 5, amino acid positions.

### Example

A CHO cell culture solution containing 0.44×10⁶ cells/ml was introduced by inoculation into a 10 l perfusion reactor (Applikon) equipped with a Biosep 50 (Applikon) in a volume of 10 l and kept for 3 days while maintaining the culturing parameters. On day 4, a 0.25 fold perfusion was started. The rate of perfusion was successively increased in each case in steps of 0.25 up to the maximum of 2.5 fold and then set according to the glucose concentrations measured in each case to within the target range of the glucose concentration (0.5-1.2 g/l). The target range for the cell number was set by adapting the cell retention rate of the ultrasound device and by exporting corresponding amounts of cell-containing culture medium. The other fermentation conditions were as follows:
Inoculum: 0.44×10⁶ cells/ml
Basic medium: HyQPF CHO Liquid Soy from HyClone/Perbio
Reactor volume: 10 l
pH: 7.2
Temperature: 37° C.
Oxygen partial pressure: 35%
Stirring speed: 200 rpm
Cell retention: Biosep 50
Fermentation time: 47 days

The basic culture medium used was enriched with protein hydrolyzates (Yeastolate from Becton Dickinson, HyPEP SR3 from Kerry Bio Science) and trace elements (CHO 4A TE Sock from Lonza).

The fermentation was recorded in respect of the analytical parameters EPO content, glucose content, glutamine content, vital cell content (absolute and relative), cell retention and perfusion. The data are summarized in FIGS. 1 to 4.

In the course of the fermentation, 776 l were harvested containing a total amount of 12 g of crude EPO. The average productivity was 15 μg/ml with an average number of cells of 1.6×10⁷ cells/ml, and the maximum number of cells was 2.6×10⁷ cells/ml with a maximum productivity of more than 30 μg/ml. The specific productivity per cell and day was 1.4 pg and the average rate of perfusion was 1.9. The vitality of the cells was between 76 and 98%. The average cell retention by means of the Biosep system was 85% (7-97%).

The harvests produced were made cell-free by filtration and subjected to a work-up and purification process known to the skilled worker, which consists of 3 to 4 chromatographic steps.

The result revealed that the process of the invention is capable of producing surprisingly high yields of an erythropoietin which has an extremely high proportion of that EPO which meets the legal requirements for medicaments, in particular in respect of its degree of glycosylation and sialylation and isoform distribution.

### DESCRIPTION OF THE FIGURES

FIG. 1 depicts the time course of the glucose concentration and EPO productivity (pg EPO/ml), respectively, in the culture supernatant obtained by the process of the invention.

FIG. 2 depicts the time course of the number of vital cells (vit. ZZ) and of EPO productivity in the culture supernatant and of the perfusion according to the process of the invention.

FIG. 3 depicts the time course of the percentage of vital cells with regard to total cells in the culture supernatant and of the percentage of cells retained by the process of the invention.

FIG. 4 depicts the time course of the lactate concentration and glutamate concentration, respectively, in the culture supernatant obtained by the process of the invention.

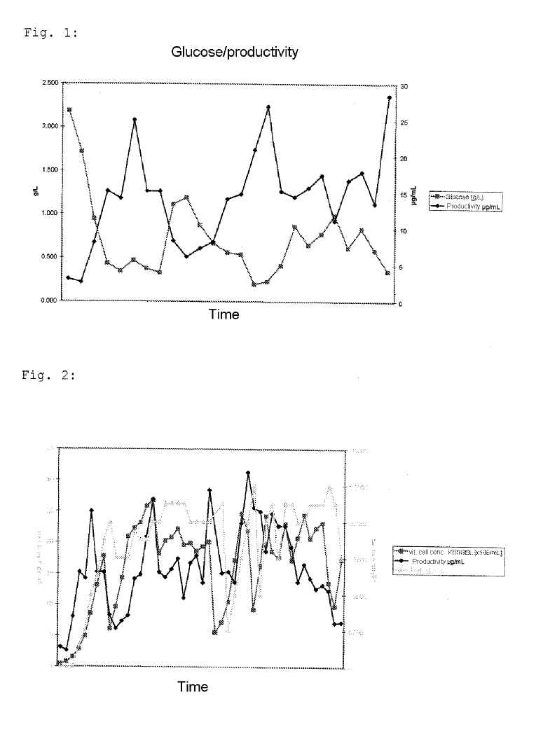

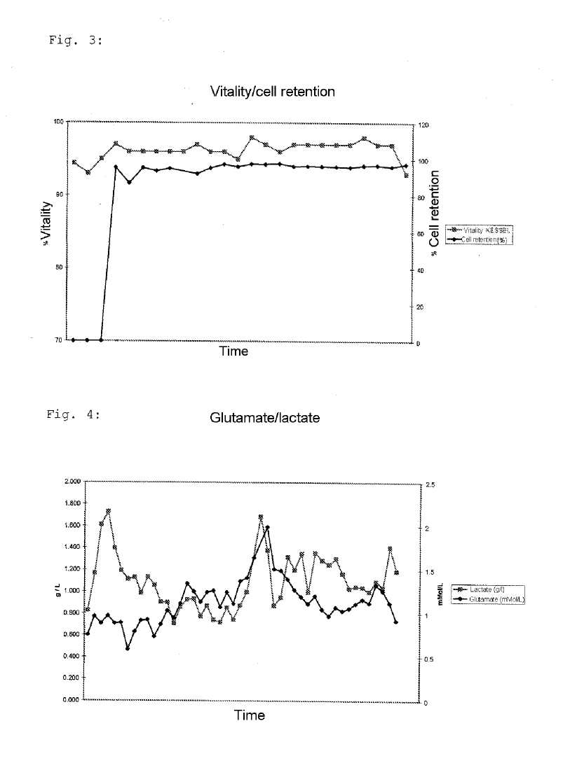

1-15. (canceled)

16. A process for the continuous fermentative production of erythropoietin, comprising culturing eukaryotic erythropoietin-producing cells in a perfusion reactor with retention of the cells to produce a culture supernatant, wherein the glucose concentration in the reactor is adjusted via the rate of perfusion of the culture medium and the number of cells in the reactor is adjusted via the rate of cell retention, in each case within a predetermined range.

17. The process of claim 16, wherein:
a) the rate of perfusion of the culture medium is adjusted as a function of the glucose concentration in the reactor within predetermined ranges, and
b) the rate of cell retention of a cell retention device is adjusted as a function of the cell density in the reactor within predetermined ranges, and/or a particular cell density in the reactor is adjusted at intervals by exporting defined amounts of cell-containing culture medium out of the reactor.

18. The process of claim 16, wherein the erythropoietin produced by said process is a variant of wild-type human erythropoietin, comprising no more than 10 amino acid substitutions, deletions or additions.

19. The process of claim 16, wherein the erythropoietin produced by said process is a variant of wild-type human erythropoietin comprising no more than 1 amino acid substitution, deletion or addition.

20. The process of claim 16, wherein the glucose concentration in the culture supernatant is adjusted within a range from 0.05 to 1.5 g/l, and the number of cells is adjusted within a range from 0.5×10⁷ to 5.0×10⁷ cells/ml.

21. The process of claim 16, wherein the eukaryotic erythropoietin-producing cells are mammalian cells.

22. The process of claim 16, wherein the eukaryotic erythropoietin-producing cells are Chinese hamster ovary cells.

23. The process of claim 16, wherein said eukaryotic erythropoietin-producing cells are retained in said perfusion reactor using an ultrasound cell retention system.

24. The process of claim 16, wherein the rate of perfusion of the culture medium and the cell retention rate are adjusted to increase the relative proportion of cells which are in their exponential growth phase.

25. The process of claim 16, wherein pH, temperature, oxygen partial pressure, stirring speed and composition of the culture medium fed into said perfusion reactor are kept constant over the entire course of the fermentation.

26. The process of claim 16, wherein at least 10 mg of erythropoietin/l of fermentation supernatant is produced.

27. The process of claim 16, wherein at least 30 mg of erythropoietin/l of fermentation supernatant is produced.

28. The process of claim 16, wherein the average specific productivity per cell per day is at least 0.5 pg, of erythropoietin.

29. The process of claim 16, wherein the average specific productivity per cell per day is at least 1.4 pg, of erythropoietin.

30. The process of claim 16, wherein the average vitality of the cells is at least 70%.

31. The process of claim 16, wherein the rate of perfusion during fermentation is between 0.5 and 3.

32. The process of claim 16, wherein said process continues for a period of at least 10 days.

33. The process of claim 18, wherein the glucose concentration in the culture supernatant is adjusted within a range of from 0.25 to 1.25 g/l,

34. The process of claim 18, wherein the number of cells in the fermentation reactor is adjusted within a range from 1.0×10⁷ to 4.0×10⁷ cells/ml of fermentation medium.

35. The process of claim 18, wherein the glucose concentration in the culture supernatant is adjusted within a range of from 0.5 to 1.0 g/l. and the number of cells in the fermentation reactor is adjusted within a range of from 1.5×10⁷ to 3.0×10⁷ cells/ml of fermentation medium.
